(19) United States
(12) Patent Application Publication
(10) Pub. No.: US 2004/0028744 A1
(43) Pub. Date: Feb. 12, 2004

(54) Mucoadhesive composition

(76) Inventors: Sauwaluxana Tongaree, Glen Allen, VA (US); Hanife Akin, Corona, CA (US); Kristen Marie Gladysz, Mechanicsville, VA (US); Khawla Abdullah Abu-Izza, Phoenixville, PA (US)

Correspondence Address: HUNTON & WILLIAMS, Intellectual Property Department, 1900 K Street, N.W., Washington, DC, 20006-1109, US

(21) Appl. No.: 10/458,148

(22) Filed: Jun. 10, 2003

Related U.S. Application Data
(63) Continuation-in-part of application No. 10/173,251, filed on Jun. 17, 2002.

(51) Int. Cl. A61K 9/14

(52) U.S. Cl. 424/487

(57) ABSTRACT

The invention is directed to a topical medicament-containing composition which exhibits excellent bioadhesive properties suitable for application to the human body including to mucous membranes and which has improved clarity and low temperatures storage properties.

This application is a Continuation-in-Part of U.S. patent application Ser. No. 10/173,251 filed Jun. 17, 2002, entitled “Mucoadhesive Composition,” the contents of which are incorporated herein in their entirety to the extent that it is consistent with this invention and application.

### BACKGROUND OF THE INVENTION

1. Field of the Invention

This invention relates to an improved topical composition. In particular, the composition exhibits bioadhesive properties which make it suitable for application to the human body, including to mucous membranes. Such properties of the composition keep the composition in contact with the affected area for several hours, resulting in a period of prolonged administration of any medicament(s) contained therein.

2. Description of Related Art

A large percentage of the population is at one time or another in need of a topically applied medicament. However, adhesion to the surface of the human body has proven to be difficult to achieve due to the presence of body oils and perspiration. Mucous membranes have been found to be a particular difficult area to achieve long-term adhesion due to the presence of increased levels of moisture and the sensitivity of such areas to chemical irritants.

U.S. Pat. No. 5,081,158 discloses a long lasting film forming composition for mucosal application. The film forming polymer used is hydroxypropyl cellulose. Organic acids such as tannic acid and salicylic acid are contained in the composition to esterify the polymer. Boric acid is further present to aid in polymer cross linking. When the composition is applied to oral mucosa, a tough film forms upon evaporation of the composition's solvent. However, the composition suffers from the presence of residual levels of free organic acids that do not participate in the esterification or cross linking reactions. These acids often cause irritation, especially if in contact with mucous membranes. Another difficulty is the limited bioadhesion exhibited by the polymer. While the polymer film may maintain its physical strength for several hours, it often detaches from the mucosa long before the film fails.

U.S. Pat. No. 5,885,611 discloses an oral gel that forms an adherent oral bandage upon evaporation of the composition's solvent. Ethylcellulose is utilized as the film-forming polymer. While ethylcellulose forms a film having good strength, the film does not exhibit good bioadhesion qualities. Therefore, such films typically do not adhere very long, often failing in less than one hour.

### OBJECTS OF THE INVENTION

It is an object of the present invention to develop a topical medicament-containing composition exhibiting good film-forming properties and good bioadhesive properties, even to mucous membranes.

It is still another object of the present invention to develop a topical composition which does not contain potential irritants such as unreacted organic acids.

It is still a further object of the present invention to develop a topical medicament-containing composition in which increased levels of the active medicament component may be incorporated.

### SUMMARY OF THE INVENTION

The present invention is directed to a topical medicament-containing composition. The composition comprises at least one medicament, menthol, a film forming composition comprising polymethacrylate, a mucoadhesive polymer, a solvent therefor and a mucoadhesive polymer. The topical medicament-containing composition exhibits good film-forming properties and good bioadhesive properties, even to mucous membranes; does not contain potential irritants such as unreacted organic acids; and provides for incorporation of increased levels of the active medicament component. Further, the medicament-containing composition of the present invention has improved clarity and low temperature stability properties.

### DESCRIPTION OF THE INVENTION

This invention relates to an improved medicament-containing composition comprising at least one medicament, menthol and a film forming mucoadhesive vehicle comprising polymethacrylate, a solvent therefor, and a mucoadhesive polymer. The choice of the solvent is not critical so long as it is suitable both for use with polymethacrylate and in topical medicament-containing (or pharmaceutical) compositions.

In U.S. patent application Ser. No. 10/173,251 we described a medicament-containing gel or liquid dosage form that forms a long lasting film upon application to mucous membranes. The gel or liquid comprises a polymethylmethacrylate (e.g., Eudragit™ RS PO) as the film-forming polymer in addition to a mucoadhesive polymer, such as Carbopol™ A medicament such as benzocaine, for example, may be used in the composition.

The invention of this application includes the use of menthol to enhance the clarity of polymeric gels comprising film forming polymer such as polymethacrylate, in ethanol solvent, a mucoadhesive polymer, such as Carbopol™, and a medicament. Carbopol™, also known as Carbomer, is an alkyl acrylate crosscopolymer. Pharmaceutical grades of Carbopol™ (Carbopol™ 934P, 971P, 974P and 980) may form slightly hazy alcoholic gels particularly in such compositions with higher levels of medicament or under cold storage conditions (e.g. 4° C.). While not wishing to be bound by theory, it is believed that the haziness is the result of the formation of a dispersion of components not solubilized.

Surprisingly, the inventors found that adding menthol to Carbopol™-based alcoholic gels significantly enhances the clarity of the gels, especially gels in which higher levels of active pharmaceutical are used (e.g., 15-25% benzocaine or higher, for example). Improved clarity enhances the product appearance and therefore improves the acceptability to the consumer. Moreover, a clear product is less likely to crystallize at low temperatures (e.g., 4° C.). Some embodiments of the invention have remained clear after several weeks of storage at 4° C. In other embodiments it was found that when menthol is added to the gel and the gel is stored at a low temperature (4° C.) for a prolonged period, crystallization may occur. However, the crystals go back in solution when the gel is allowed to stand at room temperature for a few hours. This reversal has not been observed in comparable compositions without menthol.

While not wishing to be bound to any theory, it appears that, in embodiments containing the medicament, for example benzocaine, menthol improves the clarity of the composition by increasing the solubility of medicament in the gel composition. This belief is based on the observation that the solubility of benzocaine in ethyl alcohol containing menthol is higher than the solubility of benzocaine in ethyl alcohol without menthol. It is believed that menthol can enhance the interaction between polymethacrylate, such as Eudragit™ RS, and a medicament, such as benzocaine. This belief is based on an interaction study conducted with a Differential Scanning Calorimeter (DSC). Further, while not wishing to be bound to any theory, it appears that one or both of the polymers may be forming a complex with the medicament drug in the presence of menthol and enhancing the clarity of the composition. This complex, if formed, does not appear to alter potency of the drug as compared to a composition without menthol.

The identity of the medicament component(s) is not critical. The medicament(s) should be suitable for topical application and stable within the claimed composition. It may be selected from a host of recognized medicaments heretofore used in topical applications. For instance, the medicament may be selected from one or more of the following general categories: local anesthetics, topical analgesics, antiseptic/antibacterial agents (or compounds), anti-inflammatory agents (or components), antiviral agents (or components), anti-fungal agents and mixtures thereof. Examples of local anesthetics include benzocaine, benzyl alcohol and lidocaine. Examples of antiseptic/antibacterial compounds include benzalkonium chloride, benzalkonium chloride, and cetylpyridinium. Examples of anti-inflammatory components include ibuprofen and ketoprofen. Examples of antiviral components include acyclovir. Examples of anti-fungal agents include miconazole and clotrimazole.

The concentration of the medicament(s) will, of course, vary according to their approved dosing levels and their solubility in the vehicle component of the claimed invention. For instance, benzocaine, a preferred topical anesthetic, may be present in amounts generally ranging from about 5% to about 25% on a weight percentage basis relative to the total composition. More preferably, benzocaine may be present in amounts ranging from about 10% to about 20% on the same basis. Most preferably, benzocaine may be present in an amount of about 20% on the same basis. The term “weight percentage basis relative to the total composition” means weight percentage based on the topical medicament-containing composition.

Lidocaine, another preferred topical anesthetic, may be present in amounts generally ranging from about 2% to about 10% on a weight percentage basis relative to the total composition. More preferably, lidocaine may be present in amounts ranging from about 2% to about 5% on the same basis. Most preferably, lidocaine may be present in an amount of about 5% on the same basis.

Benzalkonium chloride, a topical antiseptic, may be present in amounts generally ranging from about 0.005% to about 0.15% on a weight percentage basis relative to the total composition. More preferably, it may be present in amounts ranging from about 0.01% to about 0.02% on the same basis.

Ibuprofen, an anti-inflammatory, may be present in amounts generally ranging from about 1% to about 20% on a weight percentage basis relative to the total composition. More preferably, it may be present in amounts ranging from about 5% to about 10% on the same basis.

As stated above, the vehicle of the claimed composition comprises polymethacrylate as the film-forming polymer. The polymethacrylate polymer may be present in amounts generally ranging from about 1 to 30% by weight relative to the total composition. Preferably, it is present in amounts ranging from about 10 to about 20% by weight on the same basis. Most preferably, it is present in an amount of about 15% on the same basis. Preferred in the practice of the present invention is the use of Eudragit™ RS PO, a product manufactured by Rohm, which is a copolymer of acrylate and methacrylates with quaternary ammonium group as a functional group. Eudragit™ RS PO is insoluble in water. As a result, when the composition is applied to the oral mucosa and allowed to dry, the formed film will not dissolve in saliva and therefore lasts longer.

The vehicle also contains a solvent suitable for the polymethacrylate polymer. The choice of the solvent is not critical so long as it is suitable both for use with polymethacrylate and in topical medicament-containing (or pharmaceutical) compositions. If the medication is intended for use on canker sores, the solvent should be suitable for use on oral mucosa. The use of ethyl alcohol is preferred. The solvent may be present in amounts generally ranging from about 20 to 95% by weight relative to the total composition. Preferably, it is present in amounts ranging from about 30-70% by weight on the same basis. Most preferably, it is present in an amount of about 55-65% on the same basis.

The claimed composition also includes at least one mucoadhesive polymer that also acts as a viscosity modifying agent. Examples of such polymers (which are also viscosity modifying agents) include acrylic acid polymers (such as Carbopol™ 940, also known as Carbomer™ 940, Carbopol™ 934P, Carbopol™ 971, Carbopol™ 974P and Carbopol™ 980, products of BF Goodrich), methyl vinyl/maleic acid copolymers (such as Gantrez™ S-97, a product of International Specialty Products), polyvinyl pyrrolidone also known as povidone (such as Plasdone™ K-90, a product of International Specialty Products). These polymers impart relatively high viscosity to the composition at relatively low concentrations. They may therefore be incorporated into the claimed composition is amounts ranging from about 1% to about 5% by weight relative to the total composition. Preferably, they are present in gels in amounts ranging from about 1.5-3.5% by weight on the same basis. Most preferably, they are present in an amount of about 2-3% on the same basis. These viscosity modifying agents further act to improve the film adhesion of the composition to mucous membranes. The preferred mucoadhesive/viscosity modifying agent is Carbopol™. The preferred grades are Carbopol™ 934P, 971 P, 974P and 980 and the most preferred grade of Carbopol™ is Carbopol™ 980. The preferred level of Carbopol™ 980 is 2-3% by weight of the total composition. Most preferably, it is used in an amount of about 2.5% on the same basis.

The claimed composition includes menthol in an amount of about 0.01% to about 10% by weight relative to the total composition. Preferably, menthol is present in the composition in amounts ranging from about 2% to about 6% by weight on the same basis. Most preferably menthol is present in an amount of about 3 to about 4% by weight on the same basis.

The claimed composition may further comprise excipients such as plasticizers, flavorings, sweeteners and/or colorants. Examples of plasticizers include triethyl citrate, polyethylene glycol and glycerin. Such plasticizers are present in the composition in amounts generally ranging from about 1% to about 12% by weight relative to the total composition. For example, glycerine can be present in the amount of 1-5% by weight of the composition, preferably in the amount of 2.5% on the same basis. Polyethylene glycol and triethyl citrate can be used in the amount of about 5 to about 12%, preferably in the amount of 8%.

The claimed composition is prepared by conventional techniques wherein the polymethacrylate component, the mucoadhesive polymer (e.g., Carbopol™), menthol, optional ingredients (such as sweeteners, and/or colorants) and the medicament(s) are dissolved in the solvent and optional liquid ingredients such as plasticizers and flavors. The resulting mixture is subjected to a conventional mixing operation to evenly disperse the components.

While the viscosity of the claimed composition may vary widely depending upon its final intended application, it is preferred that it possess a viscosity of about 5000 to about 25000 cps. This can be attained through variation in the amount of the mucoadhesive polymer, which also serves as a viscosity builder.

The composition may be applied topically to a desired portion of an animal, such as a human patient, for example to the mucosa of the animal.

Upon application, the solvent rapidly evaporates leaving a tough adhesive film that both administers the medicament contained therein and acts as a protective barrier against irritants such as food and beverages. The composition may be applied on an “as needed” basis. As a general guideline, it should be administered about every 4-6 hours. The claimed composition further acts as a sustained release dosage form for the medicament, thereby prolonging the treatment intervals.

The following Examples are offered to illustrate the claimed method and its practice. They should not however be construed in any way as a limitation to the scope of the invention.

### EXAMPLE 1

A mixture having the following composition was prepared:

| INGREDIENT | 90 W/W |
|---|---|
| Benzocaine | 20.0 |
| Carbomer 980 | 2.5 |
| Eudragit ™ RS PO | 15.0 |
| Glycerin | 3.0 |
| Menthol | 4.0 |
| Opatint | 0.20 |
| Mint flavor | 2.00 |
| Ethyl alcohol | 53.3 |

The resulting mixture was placed in a stainless steel beaker and subjected to mixing conditions through the use of an air driver mixer for approximately two hours. A smooth clear gel having a viscosity of about 14,200 cps resulted.

### EXAMPLE 2

| INGREDIENT | % W/W |
|---|---|
| Benzocaine | 15.0 |
| Carbomer 940P | 2.5 |
| Eudragit ™ RS PO | 15.0 |
| Triethyl citrate | 8.0 |
| Glycerin | 3.0 |
| Saccharin | 0.25 |
| Menthol | 2.0 |
| Ethyl Alcohol | 54.25 |

The resulting mixture was placed in a stainless steel beaker and subjected to mixing conditions through the use of an air driven mixer for about 2 hours. A smooth, clear gel having viscosity of about 16,000 cps resulted.

### EXAMPLE 3

Low temperature storage tests were conducted. Compositions prepared without menthol and stored at 4° C. showed crystallization of active ingredients typically within about 24 hours. Restoration to room temperature did not reverse the crystallization. Some compositions of the invention containing menthol were stored at 4° C. and monitored for 5 weeks and no crystallization was observed at any time during the test period. Some embodiments of the invention containing certain flavor components, namely mint flavor, did show crystallization upon storage at 4° C., but the crystals disappeared and the composition returned to a clear state upon standing at room temperature for 24 hours.

### EXAMPLE 4

A mixture having the following composition was prepared:

| | % WT./WT. OF |
|---|---|
| INGREDIENT | COMPOSITION |
| Benzocaine | 20.0 |
| Carbomer 980 (acylic acid Polymer) | 3.5 |
| Eudragit ™ RS PO (poly-Methacrylate) | 20.0 |
| Triethyl citrate | 8.0 |
| Glycerin | 3.0 |
| Saccharin (sweetener) | 0.25 |
| FD&C Red #40 (colorant) | 0.01 |
| Flavor | 2.0 |
| Ethyl alcohol | 43.2 |

The resulting mixture was placed in a stainless steel beaker and subjected to mixing conditions through the use of an air driven mixer for a duration of approximately 2 hours. A smooth, clear gel having a viscosity of about 17,000 cps resulted.

### EXAMPLE 5

The mixture having the following composition was prepared:

| | % WT./WT. OF |
|---|---|
| INGREDIENT | COMPOSITION |
| Benzocaine | 15.0 |
| Carbomer 980 (acylic acid Polymer) | 2.5 |
| Eudragit ™ RS PO (poly-Methacrylate) | 15.0 |
| Glycerin | 3.0 |
| Saccharin (sweetener) | 0.25 |
| Opatint (colorant) | 0.01 |
| Flavor | 2.0 |
| Ethyl alcohol | 62.0 |

The resulting mixture was placed in a stainless steel beaker and subjected to mixing conditions through the use of an air driven mixer for about 2 hours. A smooth, clear gel having a viscosity of about 20,000 cps resulted.

### EXAMPLE 6

The mixture having the following compositions was prepared:

| | % WT./WT. OF |
|---|---|
| INGREDIENT | COMPOSITION |
| Benzocaine | 20.0 |
| Carbomer 974 (acylic acid Polymer) | 3.0 |
| Eudragit ™ RS PO (poly-Methacrylate) | 15.0 |
| Ganthrez S-97 (copolymer of methyl | 1.5 |
| vinyl/maleic acid) | |
| Glycerin | 3.0 |
| Saccharin (sweetener) | 0.25 |
| FD&C Red #40 (colorant) | 0.01 |
| Flavor | 2.0 |
| Ethyl alcohol | 43.3 |

The resulting mixture was placed in a stainless steel beaker and subjected to mixing conditions through the use of an air driven mixer for a duration of 2 hours. A slightly hazy, smooth gel having a viscosity of about 10,000 cps resulted.

### EXAMPLE 7

The mixture having the following composition was prepared:

| | % WT./WT. OF |
|---|---|
| INGREDIENT | COMPOSITION |
| Benzocaine | 15.0 |
| Carbomer 934 (acylic acid Polymer) | 2.0 |
| Eudragit ™ RS PO (poly-Methacrylate) | 15.0 |
| Ganthrez S-97 (copolymer of methyl | 2.0 |
| vinyl/maleic acid) | |
| ethylcellulose N-22 | 2.0 |
| glycerin | 5.0 |
| Saccharin Sweetner | 0.25 |
| FD&C Red #40 (colorant) | 0.01 |
| Flavor | 2.0 |
| Ethyl alcohol | 56.8 |

The resulting mixture was placed in a stainless steel beaker and subjected to mixing conditions through the use of an air driven mixer for a duration of two hours. A slightly hazy, smooth gel having a viscosity of about 11,000 cps resulted.

### EXAMPLE 8

| | % WT./WT. OF |
|---|---|
| INGREDIENT | COMPOSITION |
| Benzocaine | 20.0% |
| Carbomer 980 (acylic acid Polymer) | 2.5 |
| Eudragit ™ RS PO (poly-Methacrylate) | 15.0 |
| Glycerin | 3.0 |
| Saccharin (sweetener) | 0.25 |
| Opatint (colorant) | 0.01 |
| Flavor | 2.0 |
| Ethyl alcohol | 57.2 |

The preparation was conducted as in the previous examples. A slightly hazy gel with viscosity of approximately 13,500 cps resulted.

### EXAMPLE 9

An animal study was conducted using 6 New Zealand white rabbits to compare the efficacy of the compositions of Example 1 and Example 5 with two (2) commercial formulations, Zilactin-B™ and Orajel™ Ultra. 0.05 ml of each product was applied to the oral mucosa of 6 New Zealand white rabbits for five successive days. The animals were inspected every 30 minutes for film presence. The average duration for example was 2.34 hours. The average duration for Zilactin-B™ and Oragel™ Ultra was 1.59 hours and 1.67 hours, respectively.

### EXAMPLE 10

A sensory study was conducted using 19 healthy volunteers to compare the composition of Example #4 with a commercial product marketed under the Zilactin-B® trademark. Each sample was applied to the oral mucosa of the volunteers in the same way and allowed to dry for 30 seconds. The mucosa was then visually inspected every hour for film presence. Six hours after application, the film of Example 1 was still present in 70% of the subjects while the Zilactin® film was present in only 57% of the subjects.

What is claimed is:

1. A topical medicament-containing composition comprising:
at least one medicament component in an effective concentration;
a film forming composition comprising polymethacrylate and a solvent therefor;
a mucoadhesive polymer; and
menthol.

2. The composition of claim 1 wherein the menthol is present in an amount ranging from about 0.01% to about 10 percent by weight of the topical medicament-containing composition.

3. The composition of claim 1 wherein the menthol is present in an amount ranging from about 2 to about 6 percent by weight of the topical medicament-containing composition.

4. The composition of claim 1 wherein the menthol is present in an amount of about 3 to about 4 percent by weight of the topical medicament-containing composition.

5. The composition of claim 1 wherein the polymethacrylate is present in an amount ranging from about 1 to about 30 percent by weight of the topical medicament-containing composition.

6. The composition of claim 1 wherein the polymethacrylate is present in an amount ranging from about 10 to about 20 percent by weight of the topical medicament-containing composition.

7. The composition of claim 1 wherein the polymethacrylate is present in an amount of about 15 percent by weight of the topical medicament-containing composition.

8. The composition of claim 1 wherein the solvent is present in an amount ranging from about 20 to about 95 percent by weight of the topical medicament-containing composition.

9. The composition of claim 1 wherein the solvent is present in an amount ranging from about 30 to about 70 percent by weight of the topical medicament-containing composition.

10. The composition of claim 1 wherein the solvent is present in an amount ranging from about 55 to about 65 percent by weight of the topical medicament-containing composition.

11. The composition of claim 1 wherein the solvent is ethanol.

12. The composition of claim 1 wherein the solvent is ethanol and is present in an amount ranging from about 20 to about 95 percent by weight of the topical medicament-containing composition.

13. The composition of claim 11 wherein the solvent is ethanol and is present in an amount ranging from about 30 to about 70 percent by weight of the topical medicament-containing composition.

14. The composition of claim 11 wherein the solvent is ethanol and is present in an amount ranging from about 55 to about 65 percent by weight of the topical medicament-containing composition.

15. The composition of claim 1 wherein the medicament is selected from the group consisting of local anesthetics, antiseptic/antibacterial agents, anti-inflammatory agents, antiviral agents, anti-fungal agents and mixtures thereof.

16. The composition of claim 1 wherein the medicament is benzocaine.

17. The composition of claim 16 wherein benzocaine is present in amounts ranging from about 5 to about 25 percent by weight relative to the weight of the topical medicament-containing composition.

18. The composition of claim 16 wherein the benzocaine is present in an amount of about 20 percent by weight relative to the weight of the topical medicament-containing composition.

19. The composition of claim 1 wherein the medicament is lidocaine.

20. The composition of claim 19 wherein lidocaine is present in amounts ranging from about 2 to about 10 percent by weight of the topical medicament-containing composition.

21. The composition of claim 19 wherein lidocaine is present in an amount of about 5 percent by weight relative to the weight of the topical medicament-containing composition.

22. The composition of claim 1 wherein the medicament is benzalkonium chloride.

23. The composition of claim 22 wherein benzalkonium chloride is present in amounts ranging from about 0.005 to about 0.15 percent by weight relative to the weight of the topical medicament-containing composition.

24. The composition of claim 22 wherein benzalkonium chloride is present in amounts ranging from about 0.01 to about 0.02 percent by weight relative to the weight of the topical medicament-containing composition.

25. The composition of claim 1 wherein the medicament is ibuprofen.

26. The composition of claim 25 wherein ibuprofen is present in amounts ranging from about 1 to about 20 percent by weight relative to the weight of the topical medicament-containing composition.

27. The composition of claim 25 wherein ibuprofen is present in amounts ranging from about 5 to about 10 percent by weight relative to the weight of the topical medicament-containing composition.

28. The composition of claim 1 wherein the mucoadhesive polymer is selected from the group consisting of acrylic acid polymers, methyl vinyl/maleic acid copolymers and polyvinyl pyrrolidone polymers.

29. The composition of claim 28 wherein the mucoadhesive polymer is present in the composition in amounts ranging from about 1 to about 5 percent by weight.

30. The composition of claim 28 wherein the mucoadhesive polymer is present in the composition in amounts ranging from about 1.5 to about 3.5 percent by weight.

31. The composition of claim 28 wherein the mucoadhesive polymer is present in the composition in an amount of about 2 to about 3 percent by weight.

32. The composition of claim 1 wherein the viscosity of the composition ranges from about 5000 to about 25000 cps.

33. A method of administering a medicament to an individual comprising topically applying to said individual the composition of claim 1.

34. The method of claim 33 wherein the composition is topically applied to the mucosa of the individual.
